(19) United States
(12) Patent Application Publication
(10) Pub. No.: US 2013/0178530 A1
(43) Pub. Date: Jul. 11, 2013

(54) Nutritional Compensation For Western-Type Diet

(71) Applicants: Lucien Harthoorn, Woerden (NL); Zeina Jouni, Evansville, IN (US); Eric Van Tol, Arnhem (NL); Robert Kleemann, Leiden (NL); Teake Kooistra, Leiden (NL)

(72) Inventors: Lucien Harthoorn, Woerden (NL); Zeina Jouni, Evansville, IN (US); Eric Van Tol, Arnhem (NL); Robert Kleemann, Leiden (NL); Teake Kooistra, Leiden (NL)

(73) Assignee: MEAD JOHNSON NUTRITION COMPANY, Evansville, IN (US)

(21) Appl. No.: 13/511,497

(22) PCT Filed: Jan. 18, 2011
(86) PCT No.: PCT/US11/21497

§ 371 Date: Sep. 6, 2012

(30) Foreign Application Priority Data
Jan. 19, 2010 (EP) 10151114.5

(51) Int. Cl. A61K 31/202

(52) U.S. Cl. CPC A61K 31/202
USPC 514/560

(57) ABSTRACT

Disclosed is the use of a composition comprising docosahexaenoic acid (DHA) and/or arachidonic acid (ARA) in human subjects that are on the typical ‘Western-type” high-fat high-sugar diet. It was found, in a humanized animal model, that said composition is capable of compensating for one or more of the adverse health effects of said diet. Particularly, an effect was found in body weight reduction, without a lowering of food intake. The composition can be administered as an oral (pharmaceutical) dosage unit, as a nutritional supplement, or as a component in a food or drink.

### TECHNICAL FIELD

This disclosure relates to nutritional or pharmaceutical intervention in compensating for adverse health effects, notably body weight-related effects, of diets rich in fats and carbohydrates, such as typically found in western societies, and other developed areas of the world. Particularly, this disclosure pertains to the use of a nutritional supplement for this purpose, and to a nutritional composition, particularly a nutritional supplement for use in the prevention and treatment of said adverse health effects.

### BACKGROUND ART

Diets rich in fats and carbohydrates are increasingly being consumed by many populations in the world. This is particularly the case in western societies, such as the US and the European Union, and in other developed and developing countries throughout the world.

These diets themselves are increasingly associated with conditions such as obesity and overweight, and various health problems associated therewith, such as cardiovascular diseases, mobility disorders, type-2 diabetes mellitus, and a variety of other related disturbances or disorders. (References: Bray 2004; Parillo & Riccardi 2005; Cordain et al, 2005).

Also the Western-type high-fat high-sugar diet is frequently considered to relate to specific health conditions such as dyslipidemia, i.e. hypercholesterolemia, hypertriglyceridemia and hyperlipidemia, and impaired glucose tolerance and insulin-resistance. The effects of the diet per se are generally considered to be worsened by a general sedentary lifestyle and lack of physical exercise that large populations in the same societies are prone to.

Many solutions have been proposed, ranging from suggesting various dietary rules so as to avoid or reduce the high fat, high carbohydrate intake, to guided physical exercise methods. These methods generally suffer from serious compliance problems, or at least require great efforts to secure compliance. Also, following dietary rules (of which a wide variety of different teachings exist), generally leads to “yo-yo” effects, caused when people lose body weight by following a dietary rule for a limited period of time, and then return to their original, Western-type high-fat, high-sugar diet or intake pattern. After that they will generally gain body weight again, and frequently even end-up at a higher body weight than before starting to lose weight. Also, a large variety of dietary products (replacements or supplements) are available with the aim to help people losing body weight. These slimming products generally serve to induce a feeling of satiety, so as to reduce the intake of food. These products incur the risk that people using them may not receive a diet that is nutritionally complete.

US 2007/0203237 addresses the administration, of a therapeutically effective amount, of docosahexaenoic acid (DHA) and/or arachidonic acid (ARA) for preventing or treating obesity in a subject. To this end an amount of 15-60 mg of DHA per kg bodyweight and 20-60 mg per kg bodyweight of ARA is administered daily. The effect of the DHA/ARA as described, is to increase the amount of lean muscle in the body, and to decrease the amount of adipose tissue. The subjects generally range in age from 0 (infants) to 12 years. The described method is tested in baboon neonates.

Similarly, US 2007/0026049 refers to the administration of DHA and ARA in infants, particularly preterm infants, with the aim to increase lean body mass and reduce fat body mass.

Another disclosure in this area is US 2007/0203238, which refers to a method of reducing triglyceride levels in a subject, particularly an infant, through ARA/DHA supplementation. The purpose hereof relates to reducing the likelihood of triglyceride-linked diseases and disorders later in life. Typically, the method aims at a population that is on infant formulae, and it does not refer to effects of ARA/DHA in combination with high-fat high-sugar diets. The method is tested in baboon neonates.

US 2004/0132819 (and WO 2004/012727) describes products containing long-chain polyunsaturated fatty acids (LCPUFAs), particularly omega-3 fatty acids such as DHA, to control appetite and to help treat and/or prevent obesity and conditions of overweight, particularly in a pediatric population. The publication focuses on the effects of certain components on food intake regulation. A particularly described effect is that post-weaning feeding with ARA intake, regardless of the DHA level, results in increased food intake, whilst post-weaning feeding with DHA, regardless of the ARA level, results in reduced food intake. On this basis, the publication presents the use of omega-3 LCPUFAs such as DHA to decrease the appetite of a mammal. The present disclosure, however, starts inter alia from the acknowledgement that appetite suppression per se may not be a healthy way of provoking body weight loss, just as with the inducement of satiety by the aforementioned slimming products.

Other publications, seek to prevent obesity and other, related, disorders later in life, by administering certain compositions to infants. Thus, e.g., WO 2007/073194, presents a composition having a lipid fraction enriched in glycerophospholipids, sphingolipids and cholesterol, for administration to non-obese infants below 3 years, with the aim to prevent obesity later in life. Further, e.g., WO 2008/054208 presents certain lipids-containing compositions, inter alia based on DHA and ARA, for administration to infants between 0 and 36 months of age, with the aim to prevent and/or treat visceral adiposity and/or the accumulation of visceral fat mass to an excessive amount.

It should be noted that overweight does not always imply obesity. A person is generally considered overweight on the basis of a BMI (body mass index) of over 25, and obese on the basis of a BMI of over 30. A treatment of obesity does not always imply a treatment of overweight. On the other hand, overweight may be a pre-stage of obesity and it is therefore of potential importance to reduce weight at the stage of overweight.

In EP 1 886 680 it is recognized that in the developed world, at least, there are increasing concerns over the physiological effects of a diet rich in saturated fats and favoring the precursors of omega-6 LCPUFAs such as ARA, at the expense of the precursors of omega-3 LCPUFA, such as DHA, on the physiological omega-6: omega-3 ratio in general. The publication particularly acknowledges concerns regarding pregnant women, and proposes to administer particularly DHA to pregnant or breast-feeding women, with the aim to exert the beneficial effects thereof to the fetus or infant.

It is sometimes taught to substitute fats comprising e.g. ARA and/or DHA for a part of the fat content in a diet. Reference is made, e.g., to US 2008/269328 which pertains to health benefits relating to sources of n-3 fatty acids. In mice trials, almost half of the fat content is a so-called “designer oil” comprising ARA/DHA. The total fat exemplified is below the lower limit of what is identified as a high fat diet. Similarly, Lina et al., Food and Chemical Toxicology vol. 44, no.3, 1 Mar. 2006, pages 326-335 employs, in a diet that is not high-fat, a fat mixture comprising ARA/DHA in lieu of part of the original fat in a diet.

With the present disclosure, a different mechanism of combating overweight and related disturbances or disorders is desired, i.e. to provide a composition that helps to counteract the effects of the above-mentioned high-fat high-sugar diet. It will be understood that this is of relevance to populations that are prone to ingest a high-fat high-sugar diet, i.e. not infants or small children that are breast-feeding, on a formula, or on dedicated children's nutrition.

It would be advantageous, if a nutritional composition were provided that aids in counteracting the effects of a Western-type high-fat high-sugar diet, without having to change the diet, or to take products that induce satiety or reduce appetite.

### DISCLOSURE OF THE INVENTION

In order to better address one or more of the foregoing desires, this disclosure, in one aspect, presents the use of a composition comprising an LCPUFA selected from the group consisting of docosahexaenoic acid (DHA), arachidonic acid (ARA), metabolic precursors thereof, and combinations thereof, as a food supplement for a human subject, wherein the human subject is on a diet wherein more than 40% of the total calories is from fats, and more than 40% of the total calories is from carbohydrates.

In another aspect, this disclosure provides the use of a composition comprising an LCPUFA selected from the group consisting of docosahexaenoic acid (DHA), arachidonic acid (ARA), metabolic precursors thereof, and combinations thereof, for the purpose of compensating for one or more adverse health effects of a Western-type high-fat high-sugar diet in a human being, by administering the composition to a human subject that is on said diet.

In yet another aspect, this disclosure resides in a composition comprising an LCPUFA selected from the group consisting of docosahexaenoic acid (DHA), arachidonic acid (ARA), metabolic precursors thereof, and combinations thereof, for use in the treatment or prevention of overweight or its related disorders in human subjects that are on a Western-type high-fat high-sugar diet.

In a still further aspect, this disclosure is a method of treatment or prevention of overweight or the co-morbidities thereof, particularly cardiovascular diseases, mobility disorders, and type-2 diabetes mellitus, in human subjects that are on a Western type high-fat high-sugar diet, comprising the administration of an effective amount of a composition comprising an LCPUFA selected from the group consisting of docosahexaenoic acid (DHA), arachidonic acid (ARA), metabolic precursors thereof, and combinations thereof.

In another aspect, this disclosure presents a composition comprising an LCPUFA selected from the group consisting of docosahexaenoic acid (DHA), arachidonic acid (ARA), metabolic precursors thereof, and combinations thereof, for providing an increased HDL/LDL cholesterol ratio and a reduced VLDL-cholesterol level in a human subject that is on a diet wherein more than 40% of the total calories is from fats, and more than 40% of the total calories is from carbohydrates.

In still another aspect, this disclosure is a method of providing an increased HDL/LDL cholesterol ratio and a reduced VLDL-cholesterol level in a human subject that is on a diet wherein more than 40% of the total calories is from fats, and more than 40% of the total calories is from carbohydrates, the method comprising administering to said subject a composition comprising an LCPUFA selected from the group consisting of docosahexaenoic acid (DHA), arachidonic acid (ARA), metabolic precursors thereof, and combinations thereof.

### BEST MODE FOR CARRYING OUT THE INVENTION

The present disclosure is concerned with the adverse health effects of a ‘Western-type’ diet, also recognized as a ‘high-fat high-sugar’ diet.

The Western-type high-fat high-sugar diet is generally characterized by a high intake of processed meat, red meat, butter, high-fat dairy products, eggs, sugar and refined grains and is a common dietary habit in many developed countries and an increasing dietary pattern in developing countries.

More specifically, in this disclosure a Western-type high-fat high-sugar diet is defined in accordance with the understanding in the art, that more than 40% of the total calories in the diet is from fats, and more than 40% of the total calories in the diet is from carbohydrates.

Quantitatively, the Western-type Diet on average comprises 25-40 wt. % fat (40-70 en %, i.e. percentage of the energy provided), 40-55 wt. % of carbohydrates (40-60 en %), and 10-20 wt. % of protein (7-15 en %).

In terms of food quality, the Western-type high-fat high-sugar diet can more specifically be characterized by its high intake of saturated, trans- and omega-6 fatty acids, low content of omega-3 fatty acids, high amount of easily-digestible sugars (i.e. mono- and disaccharides) and refined grain products, and low fiber and antioxidants intake (Hu & Willett, 2002; Parillo & Riccardi, 2002; Mente et al., 2009).

Diets rich in saturated fats are diets containing, for example, lard, tallow, medium-chain triglycerides (MCT) and plant oils (e.g. palm oil, coconut oil). In such diets rich in saturated fats, generally more than 10% (wt/wt) of the diet is saturated fat, and from 30% to 50% of the total fatty acid content in the diet is saturated fat.

The aforementioned dietary characteristics promote a high glycemic response and unfavorable blood lipid profiles (Jenkins et al., 1987) and have been associated with an increased risk for type-2 diabetes mellitus, dyslipidemia, cardiovascular diseases (e.g. coronary heart failure), overweight, obesity and obesity-related disturbances (e.g. inflammatory responses) (Bray et al., 2004; Gross et al., 2004; Giugliano et al.,2006; Lairon, 2008), including childhood overweight (Ailhaud et al., 2008).

A lower cardiovascular risk profile in humans was found with lower insulin resistance (Smiley et al, 2001) and when LDL-cholesterol was substantially reduced and the HDL-cholesterol increased by more than 7.5% (Cui et al, 2009). Herein HDL stands for “high density lipoprotein” which is generally known as “good” cholesterol, and LDL stands for “low density lipoprotein” which is generally known as “bad” cholesterol.

An alternative to the Western-type high-fat high-sugar diet is either the so-called “Mediterranean-type” or “prudent” diet containing higher amounts of (non-hydrogenated) unsaturated fats as the predominant form of dietary fat, whole grains as the main form of carbohydrates, an abundance of fruits and vegetables, and adequate omega-3 fatty acids, and which have been suggested to reduce the health risks of cardiovascular disease and obesity (Hu & Willett, 2002).

The aforementioned citations are, in detail:

Ailhaud G, Guesnet P, Cunnane SC. An emerging risk factor for obesity: does disequilibrium of polyunsaturated fatty acid metabolism contribute to excessive adipose tissue development? Br J Nutr. 2008, 100, 461-470;

Bray G A, Nielsen S J, Popkin B M. Consumption of high-fructose corn syrup in beverages may play a role in the epidemic of obesity. Am J Clin Nutr. 2004, 79, 537-543;

Cui Y, Watson D J, Girman C J, Shapiro D R, Gotto A M, Hiserote P, Clearfield M B. Effects of increasing high-density lipoprotein cholesterol and decreasing low-density lipoprotein cholesterol on the incidence of first acute coronary events (from the Air Force/Texas Coronary Atherosclerosis Prevention Study). Am J Cardiol. 2009, 104, 829-834.

Giugliano D, Ceriello A, Esposito K. The effects of diet on inflammation: emphasis on the metabolic syndrome. J Am Coll Cardiol. 2006, 48, 677-685;

Gross L S, Li L, Ford E S, Liu S. Increased consumption of refined carbohydrates and the epidemic of type 2 diabetes in the United States: an ecologic assessment. Am J Clin Nutr. 2004, 79, 774-779;

Hu F B, Willett W C. Optimal diets for prevention of coronary heart disease. JAMA. 2002, 288, 2569-2578;

Jenkins D J, Jenkins A L, Wolever T M, Collier G R, Rao A V, Thompson L U. Starchy foods and fiber: reduced rate of digestion and improved carbohydrate metabolism. Scand J Gastroenterol. Suppl. 1987, 129, 132-141;

Lairon D. Macronutrient intake and modulation on chylomicron production and clearance. Atheroscler Suppl. 2008, 9, 45-48;

Mente A, de Koning L, Shannon H S, Anand S S. A systematic review of the evidence supporting a causal link between dietary factors and coronary heart disease. Arch Intern Med. 2009, 169, 659-669;

Parillo M, Riccardi G. Diet composition and the risk of type 2 diabetes: epidemiological and clinical evidence. Br J Nutr. 2004, 92, 7-19;

Smiley T, Oh P, Shane LG. The relationship of insulin resistance measured by reliable indexes to coronary artery disease risk factors and outcomes—a systematic review. Can J Cardiol. 2001, 17, 797-805.

It is to be emphasized that, whilst the terms “high-fat” and “high-sugar” (or “high-carbohydrate”) are by nature relative, the person skilled in the art is well aware of whether or not a diet can be characterized as a Western-type diet. More specifically, the more abundant form of the Western-type high-fat high-sugar diet is quantitatively characterized by a high caloric/energy density (e.g. high glycemic load) and contains on average 25-40 wt % fat, 40-55 wt % carbohydrates and 10-20 wt % protein Cordain 2005). From a qualitative perspective, the Western-type high-fat high-sugar diet is often characterized by a high cholesterol, trans- and saturated fatty acid content and a high content of refined grains (>20 en %), refined sugars (>20 en%) as well as refined fats (>20 en%), accompanied with low omega-3 fatty acid content (Cordain, 2005). In such diets rich in saturated fats, generally more than 10% (wt/wt) of the diet is saturated fat, and from 30% to 50% of the total fatty acid content in the diet is saturated fat. A specific form of the Western-type diet is recognized in the art as the “Standard American Diet” (SAD).

This disclosure is generally based on the novel and unexpected finding that ARA and/or DHA dietary supplementation, on top of a Western-type high-fat high-sugar diet, is capable of lowering body weight, whilst not affecting food intake. Metabolic precursors of ARA and/or DHA, which are well-known to the skilled person, can also be used. These precursors particularly include omega-6 fatty acid precursors, i.e. GLA (gamma-linolenic acid) and DGLA (dihomo-gamma-linolenic acid), and omega-3 fatty acid precursors, i.e. EPA (eicosapentaenoic acid) and DPA (docosapentaenoic acid).

Furthermore, the ARA/DHA dietary supplementation also has other beneficial effects when taken on top of a Western-type high-fat high-sugar diet, viz. not only on body weight development but also on liver physiology, and it can be used for treatment of dyslipidemia, i.e. hypercholesterolemia and hyperlipidemia, and insulin-resistance or type-2 diabetes mellitus. Particularly, the composition was found to be capable of providing a healthier plasma cholesterol profile and/or lower blood glucose.

This disclosure makes use of ARA, DHA, or a mixture of ARA and DHA, hereinafter referred to as the composition of this disclosure.

If the composition of this disclosure comprises a mixture, this has a ratio, by weight, of ARA:DHA typically from about 1:3 to about 9:1. In one embodiment of the present disclosure, this ratio is from about 1:2 to about 4:1. In yet another embodiment, the ratio is from about 1:1.5 to about 2:1. In one particular embodiment the ratio is about 2:1. In another particular embodiment of this disclosure, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

Preferably, the ARA and DHA are used in effective amounts of at least 129 mg/100 g food (ARA) and at least 88 mg/100 g food for DHA, or at least 25 mg/100 kcal for ARA and at least 17 mg/100 kcal for DHA.

By further preference, the composition of this disclosure is generally used so as to provide ARA in an amount of from 25 mg to 40 mg per 100 kcal food and/or DHA in an amount of from 12 mg to 60 mg per 100 kcal food. If the composition of this disclosure is a nutritionally complete formula, the foregoing amounts are per 100 kcal of such formula.

Preferred daily dosages of ARA and DHA are 50 to 750 mg/day for ARA and 50 to 450 mg/day for DHA.

The DHA and ARA can be presented in any form suitable for oral or otherwise enteral administration to a human subject. Thus, e.g., the DHA and ARA can be administered via oral dosage units, such as oral pharmaceutical dosage units, including but not limited to tablets, pills, capsules, caplets, gelcaps, oil drops, sachets, effervescent formulations, and powders.

Particularly, the composition of this disclosure is presented in the form of a dietetic or nutritional composition, suitable for human consumption. In a specific embodiment hereof, the DHA and ARA are presented as a nutritional supplement. Such a supplement can be in any form suitable for addition to foods and/or drinks, including but not limited to powders, granules, oils, oil-in-water emulsions, and pastes. In yet a further embodiment, the DHA and ARA are pre-added to a food or drink product. Thus, e.g., the ARA and DHA can be added to a dairy drink such as milk, a milk powder formulation, or yoghurt, to a dessert such as pudding, to a fruit drink, to instant soup formulations, to cheese, or to snacks such as muesli bars, candy bars, and the like.

Whilst the foregoing generally refers to oral forms of administration of the composition used in the present disclosure, the DHA and ARA can also be administered via any other enteral route of administration.

In one embodiment, the composition of this disclosure is given for the nutritional purpose of compensating for one or more of the adverse health effects of the aforementioned Western-type high-fat high-sugar diet. Herein the term “compensating” will generally be understood to have its normal meaning, viz. it refers to offsetting or counterbalancing. The term “compensating” or, for that matter, “counterbalancing” is by no means intended to refer to a full compensation or counterbalance, e.g. with reference to body weight, the compensation for the body weight increasing effect of the Western-type high-fat high-sugar diet, comprises a reduction in body weight. The reduction in body weight can be smaller than, equal to, or larger than the body weight increase to be attributed to the Western-type high-fat high-sugar diet. In the broadest sense, the use of the composition of this disclosure contributes to a compensation of one or more of the adverse health effects of taking a Western-type high-fat high-sugar diet. Preferably this contribution amounts to a full compensation.

In another embodiment, this disclosure is to be considered a method for the medical treatment or prevention of one or more of the adverse health effects of the Western-type high-fat high-sugar diet. In view of the latter, this disclosure also pertains to a composition comprising ARA and DHA as described above, for use in a method of treatment or prevention of one or more of the adverse health effects of the Western-type high-fat high-sugar diet. Such treatment or prevention will be in a human subject, said subject possibly being a patient (being on said Western-type high-fat high-sugar diet) suffering from overweight or one or more of the co-morbidities thereof, particularly cardiovascular diseases, mobility disorders, and type-2 diabetes mellitus.

The DHA and ARA according to this disclosure can be presented separately, preferably with an instruction to take them on the same day, and more preferably at about the same time. This form of presentation, i.e. separate formulations intended for simultaneous daily use, for ease of reading, is also considered hereinafter as a composition of this disclosure. Preferably, the ARA and DHA are present in one and the same formulation, i.e. present in one and the same nutritional supplement or food or drink.

In yet another embodiment, the DHA and ARA are presented in a formula intended for children such as “growing-up milk” typically aiming at children of 3 to 12 years' old. Growing-up milks, and their general composition, are known to the skilled person, and do not require elucidation here.

In some embodiments, the DHA and ARA are presented in formulations that are nutritionally complete, and which contain suitable types and amounts of lipid, carbohydrate, protein, vitamins and minerals. This disclosure does not, however, aim at substituting such formulations for normal food. Rather, as opposed to products intended to reduce appetite or other slimming products as described above, this disclosure expressly aims at allowing subjects that are on a Western-type high-fat high-sugar diet, to not change their diet, and yet be less prone to one or more health consequences of such a diet, particularly by the effect that the composition of this disclosure has on reducing the body weight of a subject on a Western-type high-fat high-sugar diet. It will be understood that the foregoing does not preclude subjects taking the composition of this disclosure to also change their diet to reduce the daily intake of saturated fat and sugar, and thus benefit from both the composition of this disclosure, and a diet that is recognized as being healthier.

The source of the ARA and DHA can be any source known in the art such as marine oil, fish oil, single cell oil, vegetable oil, egg yolk lipid, brain lipid, and the like. The DHA and ARA can be in natural form. Alternatively, the DHA and ARA can be used in refined form. Sources of DHA and ARA may be single cell oils as taught in U.S. Pat. Nos. 5,374,657, 5,550,156, and 5,397,591, the disclosures of which are incorporated herein by reference in their entirety.

This disclosure concerns the administration of the aforementioned composition comprising DHA and ARA to a human subject that is on a Western-type high-fat high-sugar diet. The expression “to be on a diet” in the context of this disclosure means that the subject generally eats on one day a week, particularly on three to five days a week, and more particularly on six to seven days a week, one or more meals that lead to the intake of levels of saturated fats and refined sugars commensurate with the common understanding in the art of the term Western-type high-fat high-sugar diet. The foregoing does not imply that this would hold for every week, but could also be the average food intake when analyzed over a period of a month, of two months, or of a longer period such as one or more quarters, trimesters, semesters, or years.

In a preferred embodiment, this disclosure more expressly relates to a direct contribution to the compensation of one or more health effects of a Western type high-fat high-sugar diet, by presenting a dietary regimen to a subject that includes one dosage of the ARA and DHA composition described above, for each day that the subject had a food intake in accordance with the Western-type high-fat high-sugar diet. More preferably, the composition is used as a daily nutritional supplement for a human subject during each week that said subject on one or more days had a food intake in accordance with the Western-type high-fat high-sugar diet. Most preferably, the composition of this disclosure is presented for permanent daily use by a subject that is on a Western-type high-fat high-sugar diet on a permanent basis. Herein “permanent” refers to an undefined period of time, and does not exclude one or more days that either the diet may be different (e.g. a subject occasionally takes a Mediterranean meal, another non-Western type high fat/high sugar diet such as a Japanese meal, or prudent diet), or the nutritional supplement may not be taken.

For use in compensating for one or more of the adverse health effects of a Western-type high-fat high-sugar diet, the composition of this disclosure is generally taken in the above-mentioned amounts effective amounts of at least 129 mg/100 g food (ARA) and at least 88 mg/100 g food for DHA, or at least 25 mg/100 kcal food of ARA and 17 mg/100 kcal food for DHA. The daily doses are strongly dependent on age and body weight, and preferably are between 50 mg/day of DHA and 50 mg/day of ARA for an infant (e.g. 6 months of age) to 1000 mg/day of DHA and 1400 mg/day of ARA for an adult human subject, and more preferably between 50 mg/day of DHA and 50 mg/day of ARA for an infant (e.g. 6 months old) to 450 mg/day of DHA and 650 mg/day of ARA for an adult woman and 550 mg/day of DHA and 750 mg/day of ARA for an adult man.

If the composition of this disclosure is administered as a medicine in the treatment or prevention of one or more of the adverse health effects of a Western-type high-fat high-sugar diet, and particularly overweight, the daily dosages can be the same or higher.

The composition of this disclosure contributes to compensating one or more of the adverse health effects of a Western-type high-fat high-sugar diet. In a preferred, most direct and measurable way, this contribution pertains to a body weight reduction in the subject, particularly without this subject being on a different (lighter or less food) diet, restrained eating or without the subject exhibiting a reduced appetite. In one preferred embodiment, the subject is a child of from 3 years old to 12 years old. In another preferred embodiment, the subject is a child (generally an adolescent) of from 12 years old to 18 years old. In yet another preferred embodiment, the subject is an adult of over 18 years old. In a particularly preferred embodiment, the adult is not of high age, i.e., below 80 years old. More particularly, said adult is not an elderly person, and is between 18 and 35 years old.

With reference to the test conducted, and without wishing to be bound by theory, the inventors consider that the body weight-reducing effect of the composition of this disclosure on human subjects that are on a Western-type high-fat high-sugar diet, is particularly seen in subjects having an age of from 3 to 80, more particularly of from 12 to 65, and most notably of from 18 to 35.

In another embodiment, the composition of this disclosure (whether or not taken by subjects thereby exhibiting a body weight reduction) contributes to compensating the plasma cholesterol profile of the subject taking the composition. Whilst the effect on body weight per se is most notably in subjects that are below high age and, particularly, that are not yet elderly persons, the life long intake of the composition also serves to provide healthier cholesterol levels, i.e. an increased HDL/LDL ratio and a reduced VLDL-cholesterol level. Herein HDL and LDL have the previously given meaning. VLDL stands for “very low density lipoprotein.”

In yet another embodiment, the composition of this disclosure (whether or not taken by subjects thereby exhibiting a body weight reduction) contributes to compensating the effects of the Western-type high-fat high-sugar diet on plasma glucose levels. This too particularly is in subjects that are below high age and, more particularly, that are not yet elderly persons.

In yet another embodiment, the composition of this disclosure contributes to compensating the effect of the Western-type high-fat high-sugar diet on liver health. Particularly, this refers to a lower liver weight.

In a particularly preferred embodiment, the composition of this disclosure is used in children of from 3 years old (36 months) to 12 years old. Effects of the administration of the composition of this disclosure can be found on body weight, better glucose homeostasis, which is recognized to prevent or delay further impaired glucose tolerance, insulin resistance and type-2 diabetes mellitus in later life, and a better lipid (i.e. cholesterol) metabolism/plasma profile, with implications for the quality of life as it lowers e.g. the cardiovascular risk profile.

It is to be understood that this disclosure is not limited to the embodiments as described hereinbefore. It is also to be understood that in the claims the word “comprising” does not exclude other elements or steps. Where an indefinite or definite article is used when referring to a singular noun e.g. “a” or “an”, “the”, this includes a plural of that noun unless something else is specifically stated.

This disclosure will be illustrated with reference to the following, non-limiting Example and the accompanying non-limiting Figures.

### DESCRIPTION OF THE DRAWINGS

FIG. 1. Body weight development of ApoE3L-mice fed a high-fat high-sugar diet. ARA/DHA dietary supplementation significantly reduced body weight over time (P<0.01, ANOVA repeated measures).

FIG. 2. Plasma total cholesterol (mM) in ApoE3L-mice fed a high-fat high-sugar diet. ARA/DHA dietary supplementation significantly reduced total cholesterol over time (P<0.01, ANOVA repeated measures).

FIG. 3. ÄKTA-FPLC-differentiation of (a) HDL- and LDL-cholesterol and (b) VLDL-cholesterol (mainly triglycerides) (mM) in plasma of ApoE3L-mice (pooled) after feeding a high-fat high-sugar diet for 16 weeks. ARA/DHA dietary supplementation reduced LDL- and VLDL-cholesterol (triglycerides) and increased HDL-cholesterol (inserted bar diagram: ARA/DHA increased the HDL/LDL ratio by about 22%).

FIG. 4. Plasma glucose (mM) after a 4-hours fasting period in ApoE3L-mice fed a high-fat high-sugar diet. ARA/DHA dietary supplementation significantly reduced fasting glucose plasma levels over time (P=0.05, ANOVA repeated measures).

FIG. 5 Mean liver weight (relative to body weight) of ApoE3L-mice at 16 weeks. ARA/DHA supplementation (stripes) showed a significant decrease in relative liver weight (P=0.01, t-test) in comparison to a high-fat high-sugar diet (black).

FIG. 6. Plasma ALAT (U/1) (alanine aminotransferase) of ApoE3L-mice at 16 weeks. In comparison to a high-fat high-sugar diet (control, black) ARA/DHA supplementation (stripes) tended top reduce ALAT (P=0.097, t-test).

### EXAMPLE

### Test Description

ARA and DHA (as currently present in infant formulae) were tested for possible beneficial effects on body weight development, dyslipidemia and insulin-resistance underlying overweight and obesity in ApoE3L-mice. These mice are a well-established humanized animal model for hyperlipidemia with overweight and mild obesity and insulin-resistance.

In the test, the ARA/DHA mixture in amounts of 0.129% w/w and 0.088% w/w, respectively (ratio 1.5:1) was added to a high-fat high-sugar diet, containing 12.2% w/w saturated fatty acids, 9.67% w/w monounsaturated fatty acids, 5.67% w/w linoleic acid (LA), 0.63% w/w alpha-linolenic acid (ALA), and 42% w/w carbohydrates, for being fed to ApoE3L-mice. The following characteristics were measured during the course of a dietary period of 16 weeks: food intake, body weight, plasma total cholesterol, total triglycerides, HDL-cholesterol, LDL-cholesterol, VLDL-cholesterol (triglycerides), fasting plasma glucose and alanine-aminotransferase (ALAT) (biomarker of liver physiology). At the end of the experiment (16 weeks) weights of different types of fat tissues (gonadal, subcutaneous, visceral and brown) and liver were determined.

The results of this study show that ARA/DHA dietary supplementation, on top of a high-fat high-sugar diet, significantly lowers body weight in ApoE3L-mice as compared to the control group with the high-fat high-sugar (including LA and ALA) diet only. The ARA/DHA containing diet did not affect food intake. Furthermore, the ARA/DHA dietary supplementation reduced plasma levels of total cholesterol and VLDL (triglycerides), and increased HDL/LDL-ratio. ARA/DHA also significantly reduced relative liver weight at 16 weeks of age, while no effects were found at the different fat-tissue weights. The ARA/DHA mixture was able to decrease fasting plasma glucose levels.

### Results:

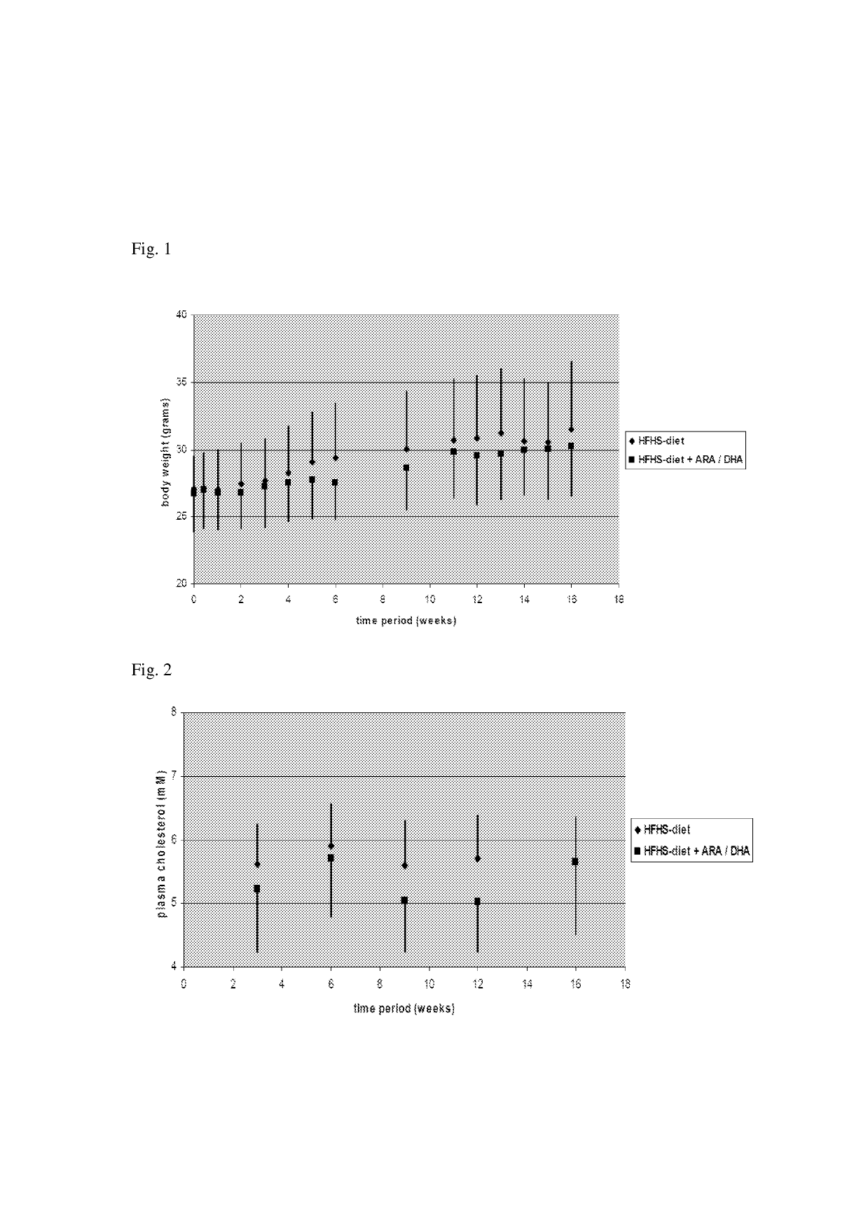

The ARA/DHA supplementation to the high-fat high-sugar diet in ApoE3L-mice showed a significantly lower body weight over time as compared to their high-fat/carbohydrate-rich (including LA and ALA) control group (FIG. 1) (P<0.01).

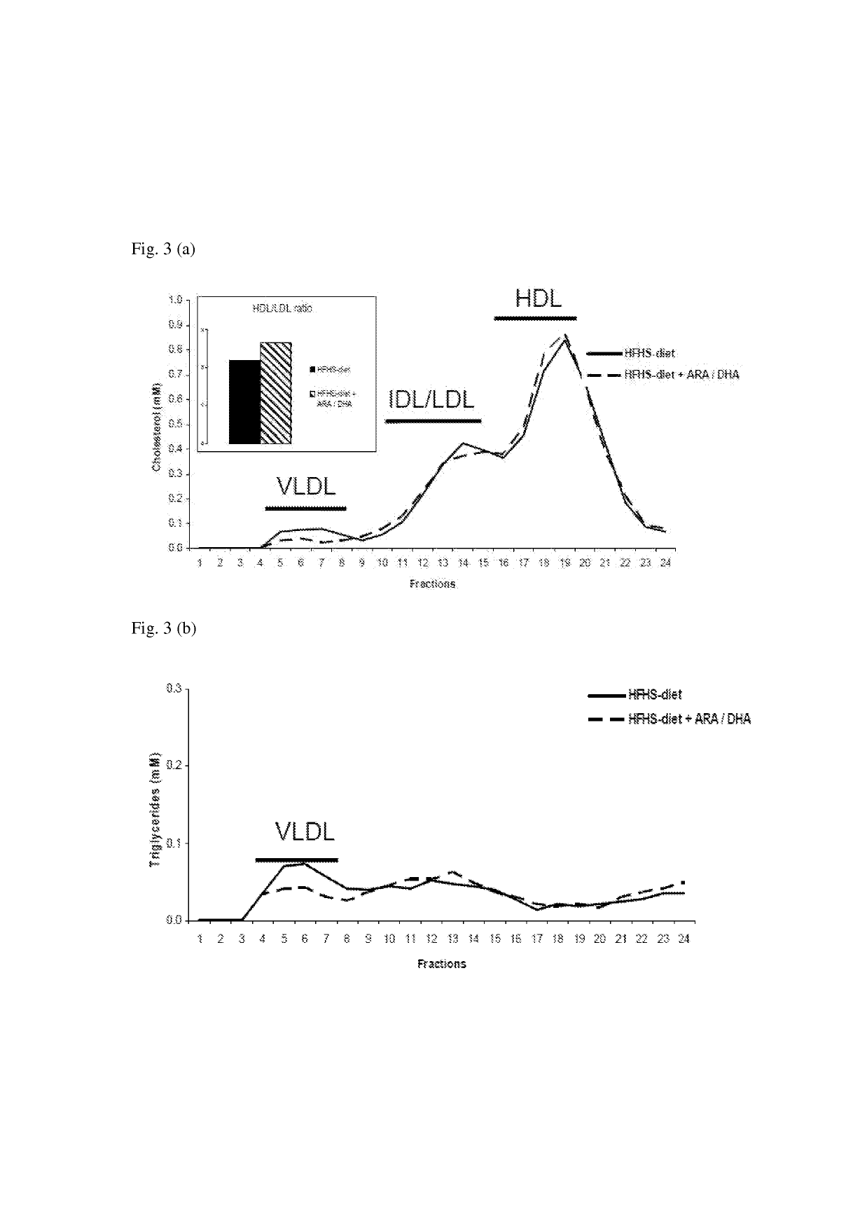

After 6 weeks, mice with the ARA/DHA supplementation had about 10% lower body weight than the control group with the high-fat high-sugar diet only, and after 16 weeks of dietary intervention the ARA/DHA fed mice still stayed at a lower body weight of about 5% in comparison to the control mice. Plasma levels of total cholesterol were significantly lower in the ARA/DHA supplemented group than in the high-fat/high-sugar control group (FIG. 2) (P<0.01). No ageing effect was found. At 9 and 12 weeks the reductions in total cholesterol were about 10%. Plasma levels of total triglycerides did not differ between the ARA/DHA treatment and the control group, however at the differentiated level after Fast Protein Liquid Chromatography (FPLC) separation of pooled plasma samples (ARA/DHA-treated group vs. control group) by means of AKTA-analysis at 16 weeks, differences were found both at the cholesterol and triglyceride profiles (FIG. 3 A, B). ARA/DHA supplementation decreased the amount of VLDL- and LDL-cholesterol particles in comparison to the pooled plasma samples of the high-fat high-sugar control group (FIG. 3(a), while HDL-cholesterol levels were higher in the ARA/DHA supplemented group. This resulted in a HDL/LDL-ratio of 2.667 in the ARA/DHA treated group vs. 2.181 in the high-fat high-sugar control group. The VLDL-profiles at the triglycerides level showed a strong reduction by ARA/DHA intake (FIG. 3(b)).

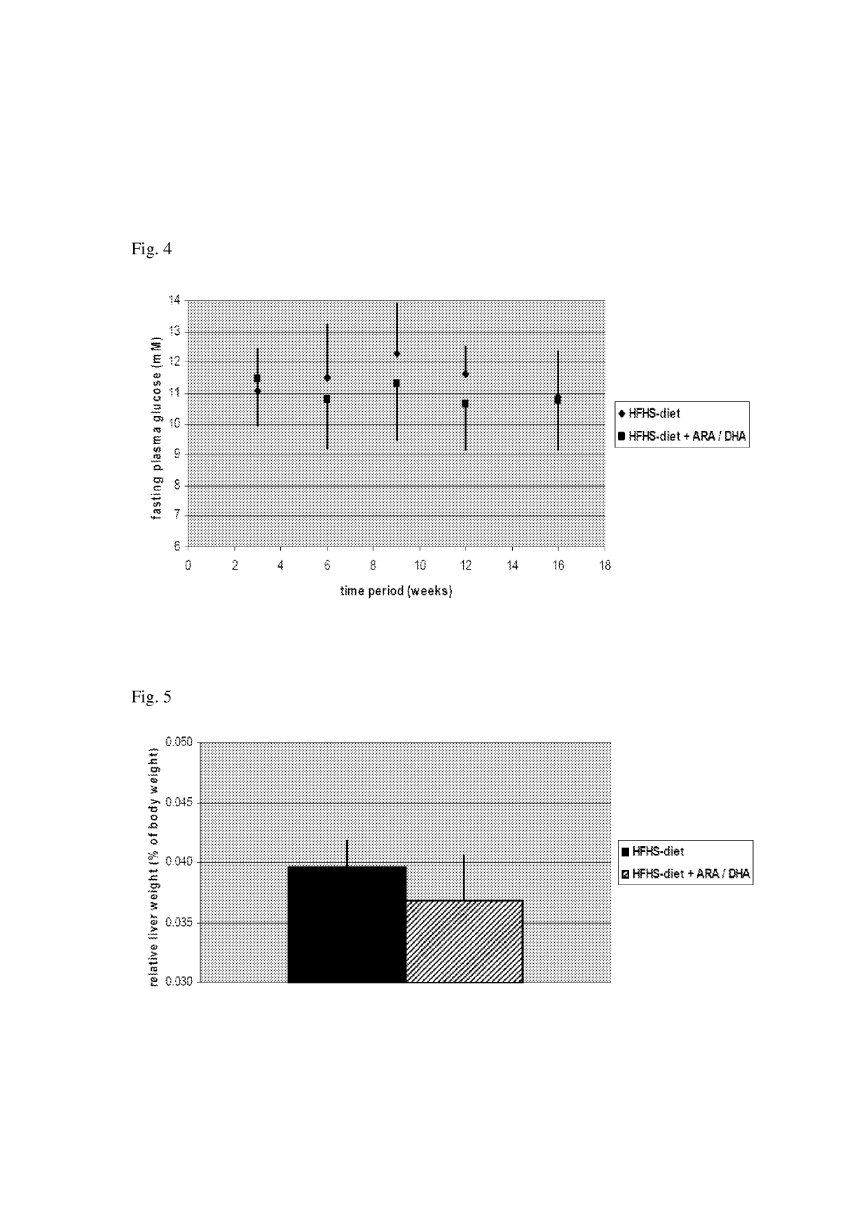

Plasma glucose levels were measured at 3, 6, 9 and 12 weeks after a 4-hours fasting period at each time point. The ARA/DHA supplemented ApoE3L-mice demonstrated a lower plasma glucose than the high-fat high-sugar control group (FIG. 4) (P=0.05). Especially at 3, 6, and 9 weeks, fasting plasma glucose levels were 6-8% lower (FIG. 4).

Relative liver weights, expressed as liver weight per body weight, of the ARA/DHA supplemented group were about 7% lower than in the high-fat high-sugar control group (P=0.01) (FIG. 5). This suggests that the high fat high-sugar diet induces fat accumulation in the liver (steatosis).

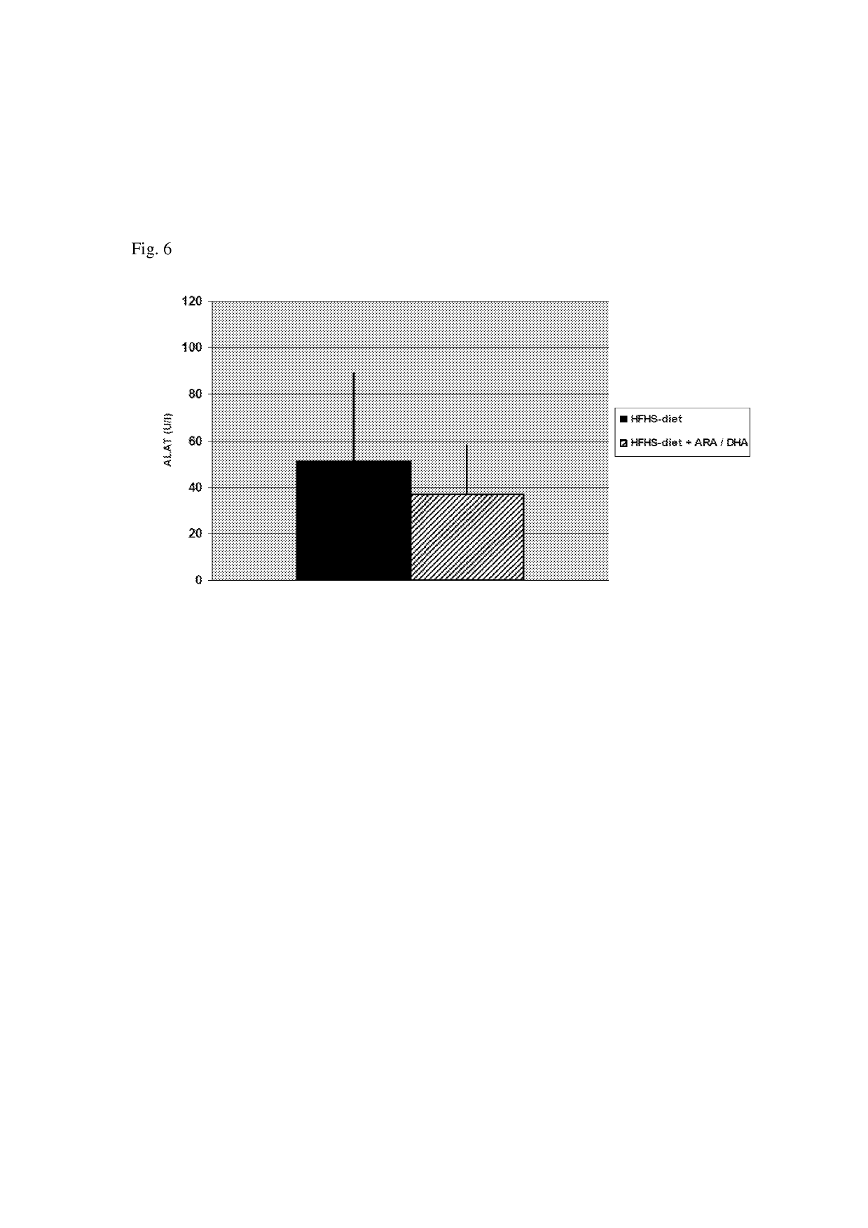

This was confirmed by the liver physiology marker ALAT (alanine amino transferase), showing the same pattern (FIG. 6). The ARA/DHA supplementation to the high-fat high-sugar diet tended to reduce ALAT.

### CONCLUSION

Under high-fat high-sugar dietary conditions, ARA/DHA dietary supplementation (0.129 w/w % and 0.088 w/w %, respectively) (ratio 1.5:1) is able to reduce body weight in ApoE3L-mice by 5 to 10%. ARA/DHA supplementation showed no changes in food intake.

ARA/DHA dietary supplementation has beneficial effects on the development of dyslipidemia in three ways, 1: it reduces hypercholesterolemia characterized by lower plasma levels of both total cholesterol and LDL-cholesterol, 2: it reduces plasma VLDL-cholesterol levels and increases the HDL/LDL ratio, and 3: it reduces hyperlipidemia shown by both a lower LDL and VLDL profile.

While a Western-type high-fat high-sugar diet may induce ectopic fat deposition in the liver (hepatosteatosis), ARA/DHA supplementation was able to reduce this as shown by lower relative liver weights.

Furthermore, dietary supplementation with ARA/DHA lowers fasting plasma glucose levels, suggesting that the ARA/DHA mixture has preventing/ameliorating properties on insulin-resistance and -sensitivity.

1. A method for supplementing the diet of a human subject which comprises providing a composition comprising an LCPUFA selected from the group consisting of docosahexaenoic acid (DHA), arachidonic acid (ARA), metabolic precursors thereof, and combinations thereof, as a food supplement for a human subject, wherein the human subject is on a diet wherein more than 40% of the total calories is from fats, and more than 40% of the total calories is from carbohydrates.

2. The method according to claim 1, wherein the daily dosage of ARA is 50 to 750 mg and/or the daily dosage of DHA is 50 to 450 mg.

3. The method according to claim 1, wherein ARA and DHA are administered in a ratio of from 1:3 to 9:1.

4. The method according to claim 3, wherein said ratio is 1:2 to 4:1.

5. The method according to claim 1, wherein DHA and ARA are administered in a single composition, preferably selected from the group of tablets, pills, capsules, caplets, gelcaps, oil drops, sachets, effervescent formulations, and powders.

6. The method according to claim 1, wherein the composition is provided in a food or drink product.

7. The method according to claim 1, wherein the diet contains on average 25-40 wt % fat, 40-55 wt % carbohydrates and 10-20 wt % protein.

8. The method according to claim 1, wherein the human subject is a child of 3-12 years of age.

9. The method according to claim 1, for the purpose of compensating for one or more adverse health effects of the diet in said subject.

10. The method according to claim 1, wherein more than 10% (wt/wt) of the diet is saturated fat, and from 30% to 50% of the total fatty acid content in the diet is saturated fat.

11. A composition comprising an LCPUFA selected from the group consisting of docosahexaenoic acid (DHA), arachidonic acid (ARA), metabolic precursors thereof, and combinations thereof, for providing an effect selected from the group consisting of (a) increased HDL/LDL cholesterol ratio and a reduced VLDL-cholesterol level, (b) a lower liver weight, (c) a lower total cholesterol, (d) a lower body weight gain, and (e) combinations thereof, in a human subject that is on a diet wherein more than 40% of the total calories is from fats, and more than 40% of the total calories is from carbohydrates, and wherein more than 10% (wt/wt) of the diet is saturated fat, and from 30% to 50% of the total fatty acid content in the diet is saturated fat.

12. A composition according to claim 11, wherein the daily dosage of ARA is 50 to 750 mg and/or the daily dosage of DHA is 50 to 450 mg.

13. A method of providing a human subject that is on a diet wherein more than 40% of the total calories is from fats, and more than 40% of the total calories is from carbohydrates, and wherein more than 10% (wt/wt) of the diet is saturated fat, and from 30% to 50% of the total fatty acid content in the diet is saturated fat, with a food supplement in order to compensate for one or more adverse health effects of the diet in said subject, wherein the food supplement is a composition comprising an LCPUFA selected from the group consisting of docosahexaenoic acid (DHA), arachidonic acid (ARA), metabolic precursors thereof, and combinations thereof.

14. A method according to claim 13, wherein the daily dosage of ARA is 50 to 750 mg and/or the daily dosage of DHA is 50 to 450 mg.

15. A method of providing an increased HDL/LDL cholesterol ratio and a reduced VLDL-cholesterol level in a human subject that is on a diet wherein more than 40% of the total calories is from fats, and more than 40% of the total calories is from carbohydrates, the method comprising administering to said subject a composition comprising an LCPUFA selected from the group consisting of docosahexaenoic acid (DHA), arachidonic acid (ARA), metabolic precursors thereof, and combinations thereof.

16. A method according to claim 15, wherein the daily dosage of ARA is 50 to 750 mg and/or the daily dosage of DHA is 50 to 450 mg.
